(19) United States
(12) Patent Application Publication
(10) Pub. No.: US 2010/0268318 A1
(43) Pub. Date: Oct. 21, 2010

(54) Prosthesis for Antegrade Deployment

(75) Inventor: Brian Glynn, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/425,271

(22) Filed: Apr. 16, 2009

(51) Int. Cl. A61F 2/06

(52) U.S. Cl. 623/1.13

(57) ABSTRACT

An endoluminal tubular prosthesis for use in an open surgical repair comprises a tubular graft having a longitudinal axis, a first tubular section having a plurality of self-expanding stents and extending along the longitudinal axis and a second stent-less tubular section extending from the first tubular section and along the longitudinal axis. The tubular prosthesis can include a plurality of tubular branching members branching therefrom for treating branched arteries without obstructing them, such as the branches from the aortic arch.

### FIELD OF THE INVENTION

The invention relates to grafts suitable for placement in a human body lumen such as an artery.

### BACKGROUND OF THE INVENTION

Tubular prostheses such as stents, grafts, and stent-grafts (e.g., stents having an inner and/or outer covering comprising graft material and which may be referred to as covered stents) have been used to treat abnormalities in passageways in the human body. In vascular applications, these devices often are used to replace or bypass occluded, diseased or damaged blood vessels such as stenotic or aneurysmal vessels. For example, it is well known to use stent-grafts, which comprise biocompatible graft material (e.g., polyester material such as Dacron® fabric, expanded polytetrafluoroethylene (ePTFE) or some other polymer) supported by a framework (e.g., one or more stent or stent-like structures) to treat vascular diseases such as aneurysms. The framework provides mechanical support and the graft material or liner provides a blood conduit. Approaches for making stent-grafts have included sewing one or more stents or annular metallic spring elements, which may have a sinusoidal configuration, to woven or laminated materials such as polyester material such as Dacron®, ePTFE, and other polymers. Many stent-grafts have a bare-spring or crown stent attached to one or both of its ends to enhance fixation between the stent-graft and the vessel where it is deployed. The bare-spring or crown stent can be referred to as an anchoring device. In treating an aneurysm, the graft material typically forms a blood impervious lumen to facilitate endovascular exclusion of the aneurysm.

In open surgical treatment of a thoracic aortic aneurysm in the ascending aorta or the aortic arch, a surgeon performs a midline sternotomy to get access to the heart and ascending aorta. The surgeon will clamp the aorta to control bleeding, The surgeon will cut an opening in the ascending aorta proximal to the aortic arch to get access to the inside of the ascending aorta and suture a surgical graft into the aorta to exclude the aneurysm. This method of accessing the ascending aorta is also used to repair the aortic valve. When surgical repair of the aortic arch is required, a surgeon may use a graft to bypass or transpose the left common carotid artery, the left subclavian artery and the brachiocephalic artery so that blood can flow from and through the graft and to the patient's head and arms. The graft, may include a branching member that may be sewn to the left common carotid artery, the left subclavian artery and the brachiocephalic artery.

When the patient's aneurysm extends from the aortic arch into the descending aorta. The surgeon will need to make another large incision on the side of the patient due to the lack of access through a the midline sternotomy

Currently, there are stent grafts that can be used to treat thoracic aneurysms of the descending aorta. The stent graft is delivered to the thoracic aorta through a catheter that is introduced onto the vasculature from the femoral artery. The catheter deploys a stent graft inside the aorta and excluding the aneurysm. However these stent grafts can not be used to exclude an aneurysm in the arch without doing a surgical hybrid procedure.

The hybrid procedure involves sewing a graft between to the brachiocephalic artery to the left carotid artery and left subclavian artery. Another stent graft is then introduced into the femoral artery deployed across the arch of the aorta starting just distal of the brachiocephalic artery. The stent-graft extends through (spans) the aneurysmal sac and beyond the proximal and distal ends thereof to replace or bypass the weakened portion of the vessel.

There remains a need to develop alternative prostheses for treating aneurysms and methods of their placement.

### SUMMARY OF THE INVENTION

The present invention involves improvements in prostheses and/or methods for their placement.

In one embodiment according to the invention, tubular prosthesis comprises a tubular graft having a longitudinal axis, a first tubular section extending along the longitudinal axis and a second stent-less tubular section being without annular stents, springs, or support members positioned about the longitudinal axis, the second tubular section extending from the first tubular section and along the longitudinal axis; and a plurality of self-expanding stents secured to the first tubular section, wherein the first tubular section forms a self-expanding stent-graft and the second tubular section forms a stent-less tubular graft.

In another embodiment according to the invention, tubular prosthesis comprises a tubular graft having a longitudinal axis, a first tubular section extending along the longitudinal axis and a second tubular section extending from the first tubular section and along the longitudinal axis; and a plurality of self-expanding stents secured to the first tubular section, wherein the first tubular section forms a self-expanding stent-graft and the second tubular section forms a tubular graft that is not self-expanding.

In another embodiment according to the invention, a tubular prosthesis comprises a tubular graft having a longitudinal axis, a first tubular section extending along the longitudinal axis and a second tubular section extending from the first tubular section and along the longitudinal axis, the first tubular section having a length of at least 50 mm and being without an annular support member; and a plurality of stents secured to the first tubular section, wherein the first tubular section forms a self-expanding stent-graft and the second tubular section forms a tubular graft that is not self-expanding.

In another embodiment according to the invention, tubular prosthesis comprises a tubular graft having a longitudinal axis, a first tubular section and a second tubular section and a plurality of tubular branching members, the first tubular section extending along the longitudinal axis and the second tubular section extending from the first tubular section and along the longitudinal axis, the first tubular section including a plurality of stents secured thereto, the second tubular section being without an annular support member, each of the plurality of tubular branching members including at least one stent secured thereto, and the plurality of tubular branching members branching from the second tubular section an being in fluid communication therewith.

In another embodiment according to the invention, tubular prosthesis apparatus comprises a tubular graft having a longitudinal axis, a first tubular section, and a second tubular section, the first tubular section extending along the longitudinal axis and the second tubular section extending from the first tubular section and along the longitudinal axis, the first tubular section having a first configuration and a second radially compressed configuration, the second tubular section forming a lumen; a plurality of tubular branching members branching from the second tubular section, each of the branching members having a first configuration and a second radially compressed configuration, each branching member forming a lumen that is in fluid communication with the lumen formed by the second tubular section; and a plurality of sleeves, each one of the sleeves surrounding one of the first tubular section and plurality of tubular branching members and restraining the first tubular section and the plurality of tubular branching members in the radially compressed configurations.

In another embodiment according to the invention, a method of treating an aneurysm comprises advancing a tubular prosthesis having a restrained first self-expanding tubular section with a plurality of stents and a second tubular stent-less section with the first tubular section in a radially compressed state through a vessel to an unexposed vessel location where the first tubular section spans a target site; unrestraining the first tubular section to allow the first tubular section to radially expand; and securing the second tubular stent-less section of the tubular prosthesis to the vessel at a location where the vessel has been exposed.

### BRIEF DESCRIPTION OF THE DRAWINGS

FIG. 1A illustrates one embodiment of a prosthesis according to the invention.

FIG. 1B illustrates the embodiment illustrated in FIG. 1A in a delivery state with a plurality of deployment mechanisms each having a restraints restraining a portion of the prosthesis in a radially compressed or reduced diameter configuration as compared to the uncompressed configuration shown in FIG. 1A.

FIG. 1C illustrates the bare frame of the core catheter shaft elements of the plurality of deployment mechanisms shown in FIGS. 1A and 1B each having hook like prosthesis tip capture elements configured to hold a end portion of the stent graft prosthesis at the end of the shaft of the implanting catheter away from the implanting physician extended in tension and fixed to the end of the respective shaft as its respective sheath is retracted.

FIG. 2A is a longitudinal partial sectional view of one restraint mechanism loaded with a section of the prosthesis of FIG. 1A.

FIG. 2B is an end view of the apparatus of FIG. 2A taken along line 2B-2B.

FIG. 2C diagrammatically shows partial deployment of a section of the prosthesis shown in FIG. 2A depicting splitting and withdrawal of the restraint, which is in the form of a sleeve.

FIG. 2D diagrammatically shows further deployment of the section of the prosthesis being deployed in FIG. 2C.

FIGS. 3A-E diagrammatically illustrates one method of using the prosthesis shown in FIGS. 2A and 2B, where FIG. 3A diagrammatically illustrates the physician's view once a midline sternotomy has been performed to expose the heart and its aortic arch, FIG. 3B diagrammatically illustrates a side view of the aorta (the descending aorta generally not being visible in the midline sternotomy) and an incision formed in the ascending aorta and the aortic arch, FIG. 3C diagrammatically illustrates the prosthesis of FIG. 1A with restraints as shown in FIG. 2B after one end of the device has been introduced through the ascending aorta incision, through the aortic arch and into the descending aorta, FIG. 3D diagrammatically illustrates partial deployment of one section of the prosthesis, and FIG. 3E diagrammatically illustrates the prosthesis fully deployed.

### DETAILED DESCRIPTION

The following description will be made with reference to the drawings where when referring to the various figures, it should be understood that like numerals or characters indicate like elements.

In one embodiment according to the invention, a tubular prosthesis comprises a tubular graft having a longitudinal axis, a first tubular section having a plurality of self-expanding stents and extending along the longitudinal axis and a second stent-less tubular section extending from the first tubular section and along the longitudinal axis. With this configuration, an antegrade approach can be used to treat an aneurysm (e.g., a thoracic aortic aneurysm that extends along the descending aorta) where a physician advances the first tubular section through an opening in an exposed vessel (e.g., an opening formed in the ascending aorta after a midline sternotomy) while the first tubular section is in a radially compressed state, and through the vessel to an unexposed vessel location (e.g., the descending aorta after a midline sternotomy) where it spans a target site where the first tubular section is allowed to expand and the second tubular section sewn to the vessel at a location where the vessel has been exposed (e.g., along the ascending aorta that has been exposed during a midline sternotomy). Other advantages will become apparent from the following description.

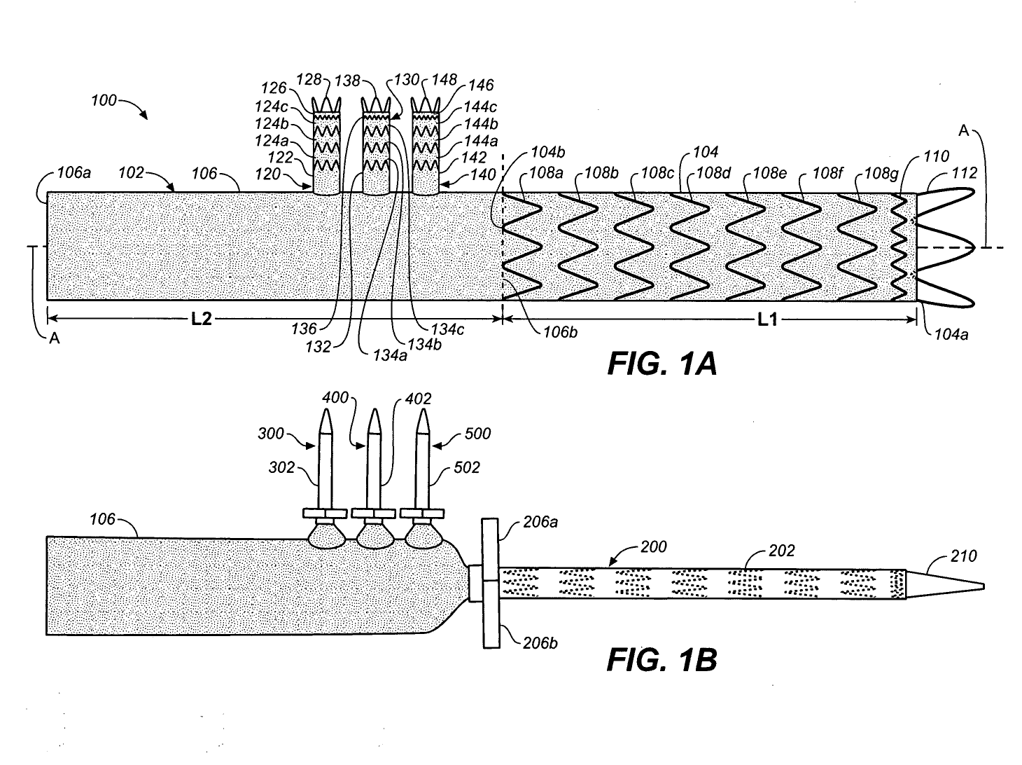

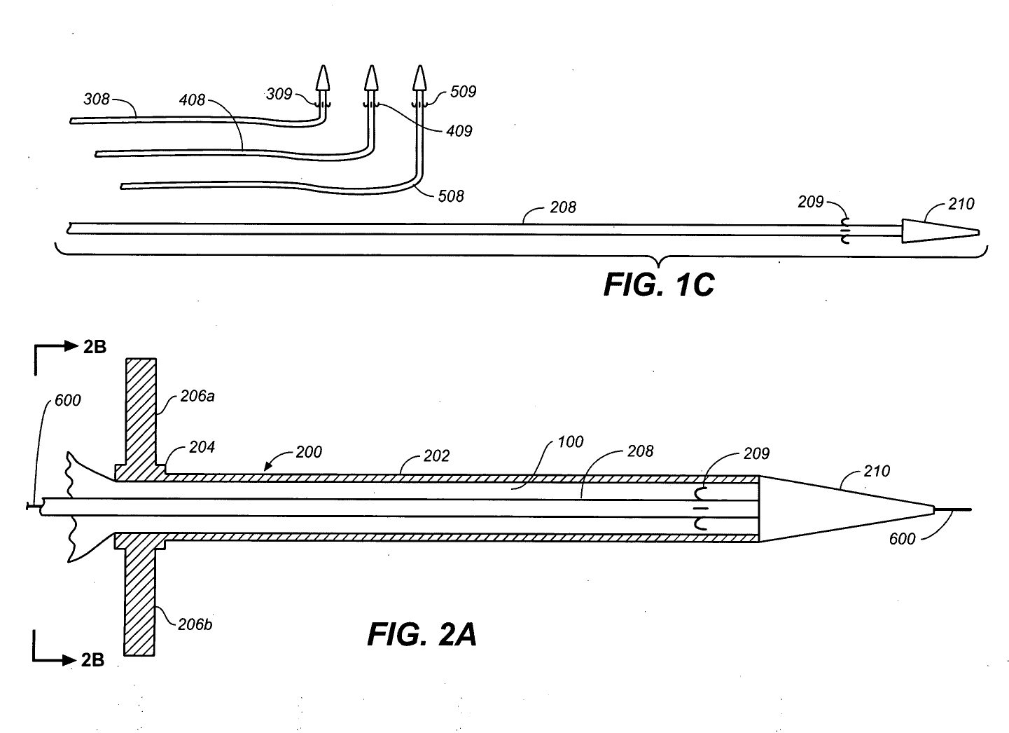

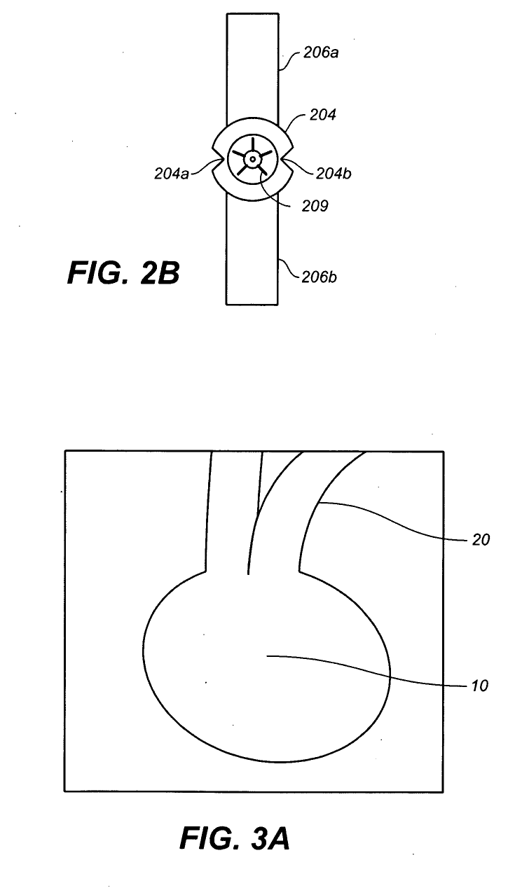

Referring to FIG. 1A, one prosthesis embodiment according to the invention is shown and generally indicated with reference numeral 100. Prosthesis 100 includes a tubular graft (member) 102 having a longitudinal axis “A” and first and second tubular sections 104 and 106. First tubular section 104 includes a plurality of stents and second tubular section 106 has no stents. In other words, first tubular section 104 forms or corresponds to a stent-graft or covered stent and second tubular section 106 forms or corresponds to a stent-less tubular graft. In the embodiment illustrated in FIG. 1A, second tubular section 106 is without any annular support such as an annular stent or an annular spring such as a sealing spring. Tubular graft 102 can be made from any suitable material such as polyester, PTFE, ePTFE, UHMWPE, PET, Kevlar® fiber, Dacron® fabric, or PEEK material. First tubular section 104 of tubular graft 102 has secured thereto stents 108a, 108b, 108c, 108d, 108e, 108f, and 108g which are secured to the graft using any known techniques such as suturing. However, more or fewer stents can be used depending on the application and desired length of section 104. First tubular section 104 also can provided with a sealing spring, such as sealing spring 110, which similarly can be sutured to the tubular graft. A bare spring such as bare spring 112, which can be referred to as a crown stent and which assists in anchoring the prosthesis in a vessel, also is optional and can be secured to first tubular section 104 adjacent to sealing spring 110. The stents, sealing spring, and bare spring are annular members that have undulating configurations and can be formed from nitinol or any other suitable material.

First tubular section 104 of tubular graft 102 has an exposed first end 104a and a blind second end 104b. Second tubular section 106 of tubular graft 102 similarly has an exposed first end 106a and a blind second end 106b. Sections 104 and 106 can be integrally formed from a single piece of graft material or separately formed and secured to one another at blind ends 104b and 106b using any known technique such as suturing or they can be interwoven. For example, first tubular section 104 can be constructed as a stent-graft (or covered stent) and then secured to tubular graft section 106, which in the illustrative embodiment is without any annular support structure such as an annular stent or an annular spring such as a sealing spring.

Prosthesis 100 also includes three tubular branch or branching members, which in the illustrative embodiment correspond to stent-grafts (or covered stents) 120, 130, and 130, branching from second tubular section 106. Tubular member 102 forms a lumen and each branch member or stent-graft 120, 130, and 130 forms a lumen that is in fluid communication with the lumen formed by tubular member 102. Stent-graft 120 includes a tubular graft 122, annular undulating stents 124a, 124b, and 124c secured (e.g., stitched) thereto, sealing spring 126 secured (e.g., stitched) thereto, and bare spring (or crown stent) 128 secured (e.g., stitched) thereto. Stent-graft 130 includes a tubular graft 132, annular undulating stents 134a, 134b, and 134c secured (e.g., stitched) thereto, sealing spring 136 secured (e.g., stitched) thereto, and bare spring (or crown stent) 138 secured (e.g., stitched) thereto. Stent-graft 140 includes a tubular graft 142, annular undulating stents 144a, 144b, and 144c secured (e.g., stitched) thereto, sealing spring 146 secured (e.g., stitched) thereto, and bare spring (or crown stent) 148 secured (e.g., stitched) thereto. Stent-grafts 120, 130, and 140 can be stitched or sutured to second tubular section 106 of tubular member 102. Although three branching members are shown secured to second tubular section 106 of tubular member 102, fewer branching members may be used depending on the application (e.g., the prosthesis can include one or no branching members).

The dimensions of the prosthesis will depend on the application. When used in antegrade deployment from the ascending aorta to the descending aorta during open heart surgery, second tubular section 106 will have a length “L2” measured along longitudinal axis “A” of at least 50 mm, which corresponds to the minimal length of the aortic arch plus an additional length to cut from outside the aorta after the first tubular section is deployed and trimmed after first end 106a of second tubular section 106 or a section adjacent thereto has been sutured to the ascending aorta. In this application, first tubular section 104 typically will have a length “L1” measured along longitudinal axis “A” of 100 mm to about 500 mm and typically will be about 200 mm. The stent-graft branching members are configured for placement in the brachiocephalic artery, left common carotid artery, and left subclavian artery and typically will have a length from about 20 mm to about 80 mm. In another example, the prosthesis is placed to treat an aneurysm or stenosis in the _superficial femoral artery _. In this example, prosthesis 100 has no branching members. A cut is made at the groin of the patient down to the femoral artery, the femoral artery is clamped, and an incision made in the femoral artery. The prosthesis is introduced into the femoral artery, advanced down the entire superficial femoral artery to the popliteal artery. In other words, the prosthesis is placed from the hip to the knee of the patient. The first tubular section restraint is released to allow the first tubular section to expand and the second tubular section, which typically will be without any annular support such as an annular stent or an annular spring such as a sealing spring, is cut outside the femoral artery, sutured to the femoral artery near the groin, and trimmed. In this application, second tubular section 106 will have a length “L2” measured along the longitudinal axis “A” of least 100 mm and up to about 1,000 mm. First tubular section 104 of tubular graft 102 (the section having stents) will have a length “L1” measured along the longitudinal axis “A” of at least 30 mm, which typically corresponds to a stent-graft having two or three stents.

Referring to FIG. 1B, prosthesis 100 is shown in a delivery state with first tubular section 104 and branching members 120, 130, and 140 radially compressed and restrained in deployment mechanisms or devices 200, 300, 400, and 500. In this embodiment, each of first tubular section 104 and branching members 120, 130, and 140 is a self-expanding stent-grafts. Each deployment mechanism or device includes a restraint, which in the illustrative embodiment is in the form of a tubular splittable sleeve 202, 302, 402, and 502, to restrain a portion of the prosthesis in a radially compressed or reduced diameter configuration for advancement through a vessel or endolumenal advancement. More specifically, splittable sleeves 202, 302, 402, and 502 restrain stent-grafts (or covered stents) 104, 120, 130, and 140 in a radially compressed state about distal end portions of inner tubes 208, 308, 408, and 508.

Referring to FIG. 1C, only the core elements of the delivery catheter elements are shown picturing the end stent capture fingers 209, 309, 409, 509. These fingers function to prevent the prosthesis section contained in the respective surrounding sleeve from moving back with the surrounding sleeve, as it is split and retracted. These fingers assure the fully extension of the graft material and stent graft element in its respective surrounding lumen.

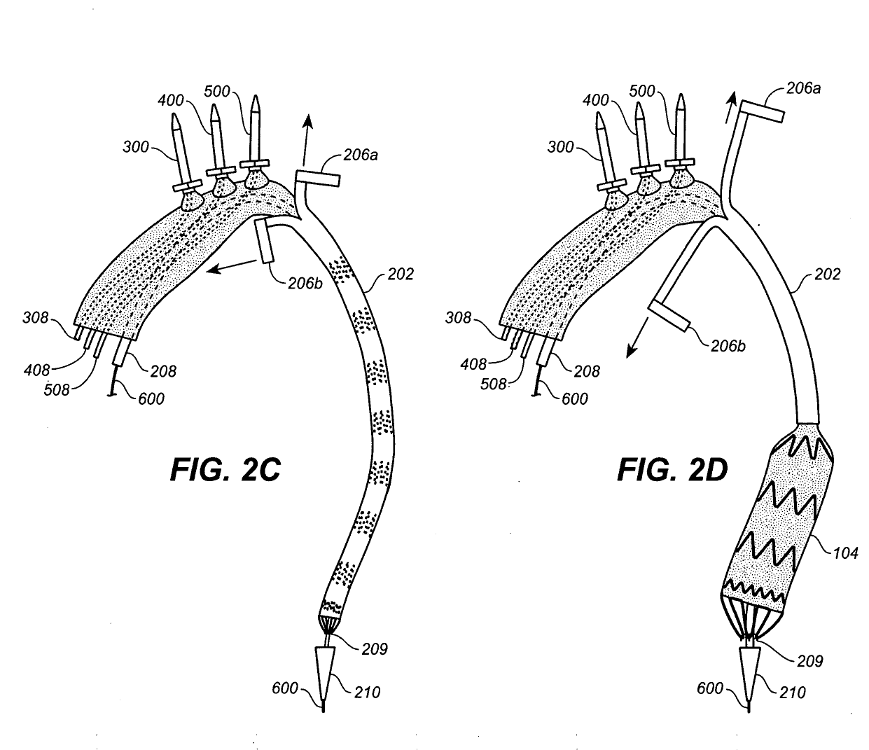

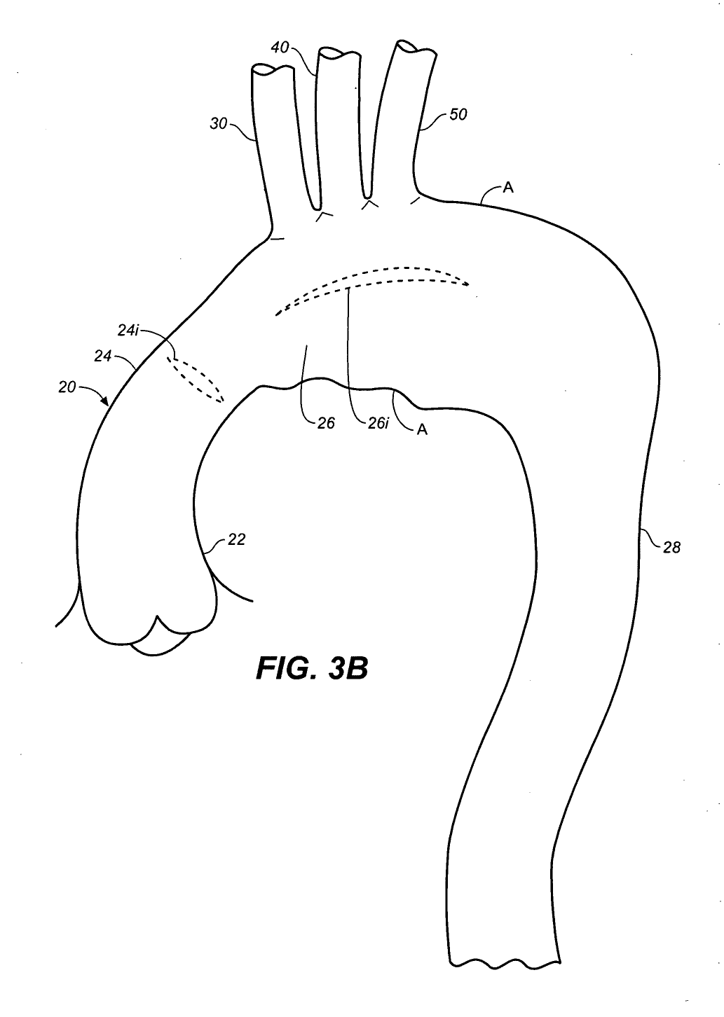

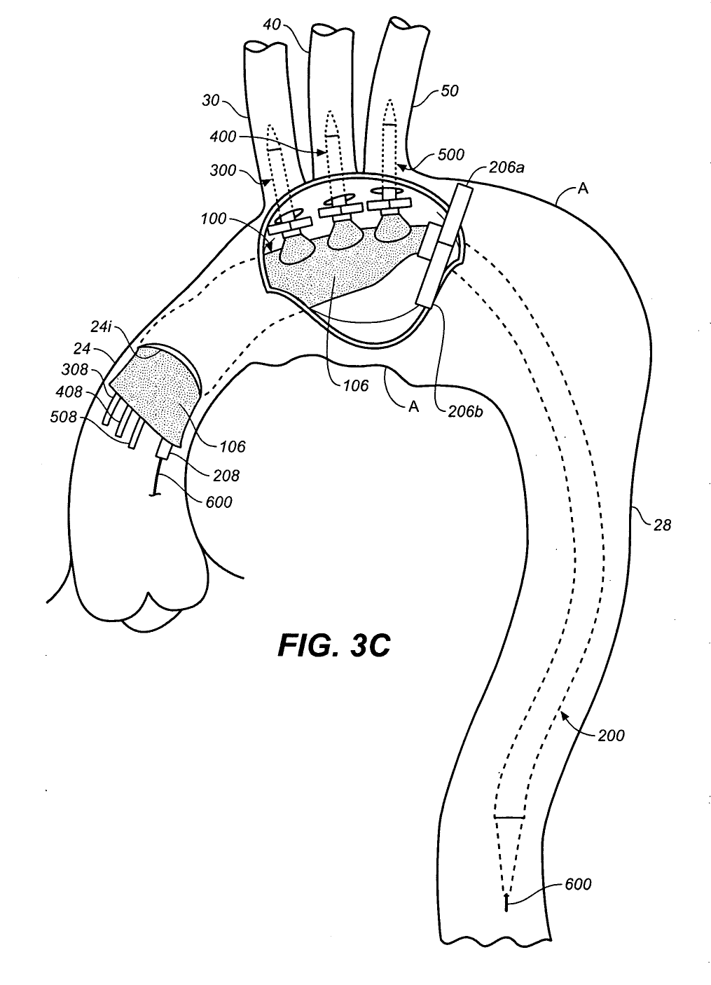

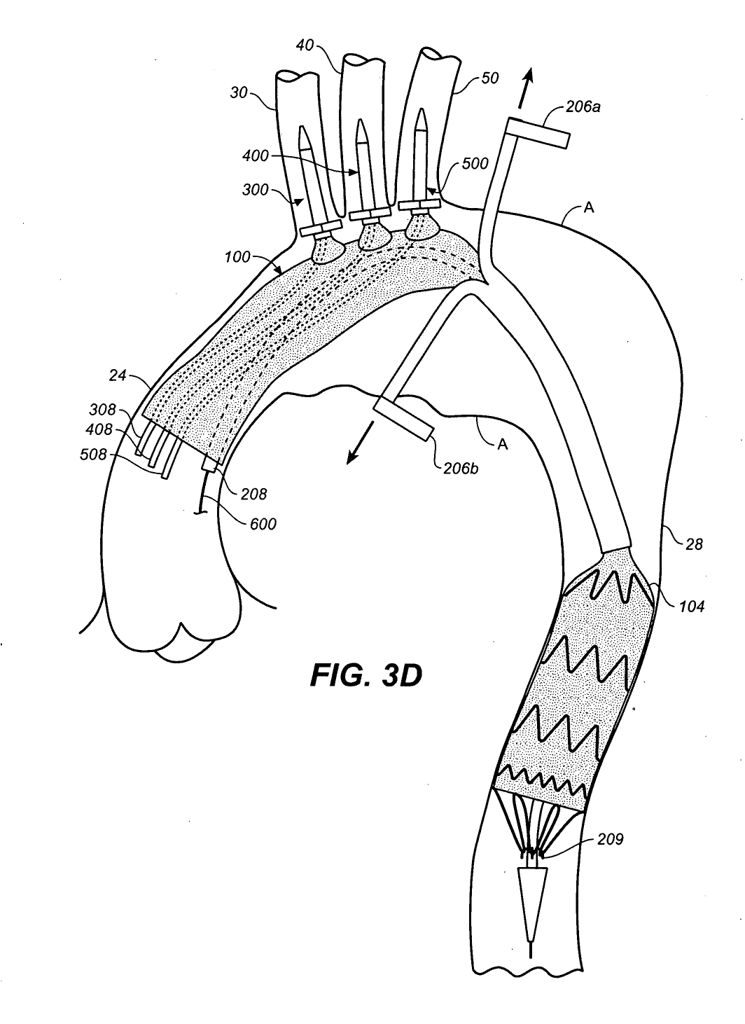

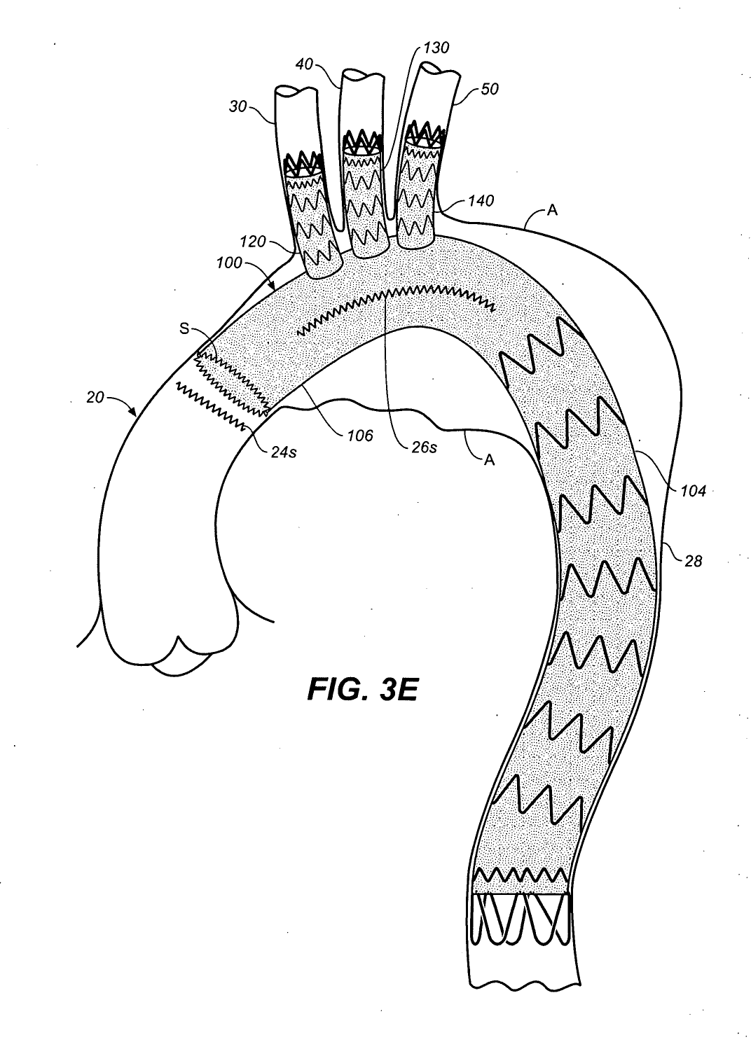

Referring to FIG. 2A, deployment mechanism or device 200 is shown in partial section. Since deployment mechanisms 200, 300, 400, and 500 all have the same construction, only deployment mechanism 200 will be described in detail. Deployment mechanism 200 includes outer splittable restraint or sleeve 202 having annular hub portion 204 extending therefrom tabs 206a and 206b extending radially from hub portion 204 all of which can be integrally formed as a single piece construction. Annular hub portion 204 has reduced thickness sections 204a and 204b that extend the full extent of hub portion 204 in the longitudinal direction. Sleeve 202 and sleeve hub portion and 206 are relatively soft plastic material such as polyethylene. so that when tabs 206a and 206b are pulled apart, the reduced sections split and the diametrically opposed splits formed in sleeve 202 continue to run along sleeve 202 as one continues to pull the tabs. An inner tube 208 is disposed inside sleeve 202 through which optional guidewire 600 can be slidably disposed. Integrally formed with tube 208 is tapered tip 210 and attached thereto are retaining fingers 209, one finger to engage each crown of an end stent of the prosthesis section to be deployed. First portion 104 of tubular member 102 is radially compressed and inserted over tube 208 with the end stent's crowns being engaged and captured by the retaining fingers 209. The first portion 104 is positioned between tube 208 and sleeve 202. Tube 208 is sufficiently long so that a physician or operator can hold tube 208 which in turn transmits a compressive force to retaining fingers 209′ while tabs 206a and 206b are pulled apart in a radial direction to maintain tube 208 stationary so that the sleeve will split and retract. FIGS. 2C and 2D are illustrative of how the sleeve is split to deploy section 104. Referring to FIG. 2C, the physician or operator holds tube 208 stationary. Another physician or operator can pull tabs 206a and 206b to simultaneously spit and retract sleeve 202 so that first tubular section 104 begins to deploy as shown in FIG. 2C. As the tabs are further pulled, the sleeve is further withdrawn as shown in FIG. 2D until the sleeve is fully split and first tubular section fully deployed (see FIG. 3E).

Referring to FIGS. 3A-E, one method of using prosthesis 100 is diagrammatically shown. FIG. 3A diagrammatically illustrates a midline sternotomy where a patient's heart 10 and a portion of aorta 20 is shown. Generally speaking, the portion of the aorta distal to the left subclavian artery (i.e., the descending aorta) is not visible or accessible for surgery from the outside thereof without turning the patient on the patient's side and cracking ribs on a side of the patient and cutting down to the descending aorta. FIG. 3B diagrammatically illustrates a side view of aorta 20, which extends from aortic root 22 and includes ascending aorta 24, aortic arch 26, and descending aorta 28, to show how prosthesis 100 will be introduced into the descending aorta to bypass an aneurysm “A,” which extends from an area proximal to the aortic arch to the region of the aorta distal to the left subclavian artery 50. The physician makes an incision 24i in ascending aorta 24 for introduction of the prosthesis into the aorta and longitudinal incision 26i in aortic arch 26, which can be flapped back so that the physician can insert the prosthesis branching members into branch arteries 30 (brachiocephalic), 40 (left common carotid), and 50 (left subclavian) and manipulate tabs 206a and 206b as will be described in more detail below. FIG. 3C diagrammatically illustrates prosthesis 100 in its delivery state as described above with reference to FIG. 1B and after it has been introduced through incision 24i with first tubular section 104, which is in deployment mechanism or device 200, positioned in descending aorta 28, and branching stent-grafts 120, 130, and 140, which are in deployment mechanisms or devices 300, 400, and 500, positioned in brachiocephalic artery 30, left common carotid artery 40, and left subclavian artery 50. If optional guidewire 600 is used, it placed through incision 24i and into the descending aorta and then prosthesis 100 is tracked over the guidewire. Referring to FIG. 3D, tube 208 is held so that it and the portion of the prosthesis contained therein to be deployed is axially restrained and tabs 206a and 206b pulled to split and retract sleeve 202. After sleeve 202 is fully split into two separate pieces and stent-graft section 104 fully deployed, sleeve 202, guidewire 600, and tube 208 are removed. Then sleeves 302, 402, and 502 are split and retracted in the same manner to release branching stent-grafts 120, 130, and 140. Sleeves 302, 402, and 502 and tubes 308, 408, and 508 are removed. The proximal end of tubular graft 102, which is the end closest to the heart, is sutured to the aorta as depicted with line “S” and the incisions sutured closed as shown in FIG. 3E. Incisions 24i and 26i are sutured closed as indicated with reference numerals 24s and 26s.

Any feature described in any one embodiment described herein can be combined with any other feature or features of any of the other embodiments or features described herein. Furthermore, variations and modifications of the devices and methods disclosed herein will be readily apparent to persons skilled in the art.

What is claimed is:

1. A tubular prosthesis comprising:
a tubular graft having a longitudinal axis, a first tubular section extending along said longitudinal axis and a second tubular section extending from said first tubular section and along said longitudinal axis; and
a plurality of self-expanding stents secured to said first tubular section, wherein said first tubular section forms a self-expanding stent-graft and said second tubular section forms a tubular graft that is not self-expanding.

2. The prosthesis of claim 1 wherein said second tubular section is without a stent.

3. The prosthesis of claim 2 further including a plurality of tubular branch members extending from said second tubular section, said first and second tubular sections forming a lumen and each tubular branch member forming a lumen, wherein each branch member lumen is in fluid communication with said lumen formed by said first and second tubular sections.

4. The prosthesis of claim 3 wherein each tubular branch member forms a self-expanding stent-graft.

5. The prosthesis of claim 4 wherein said first tubular section forms a self-expanding stent-graft.

6. The prosthesis of claim 2 wherein said second tubular section has a length measured along said longitudinal axis of at least 50 mm.

7. A tubular prosthesis comprising:
a tubular graft having a longitudinal axis, a first tubular section extending along said longitudinal axis and a second tubular section extending from said first tubular section and along said longitudinal axis, said first tubular section having a length of at least 50 mm and being without an annular support member; and
a plurality of stents secured to said first tubular section, wherein said first tubular section forms a self-expanding stent-graft and said second tubular section forms a tubular graft that is not self-expanding.

8. The prosthesis of claim 7 further including a plurality of tubular branch members extending from said second tubular section, said first and second tubular sections forming a lumen and each tubular branch member forming a lumen, wherein each branch member lumen is in fluid communication with said lumen formed by said first and second tubular sections.

9. The prosthesis of claim 8 wherein each tubular branch member forms a self-expanding stent-graft.

10. Tubular prosthesis apparatus comprising:
a tubular graft having a longitudinal axis, a first tubular section, and a second tubular section, said first tubular section extending along said longitudinal axis and said second tubular section extending from said first tubular section and along said longitudinal axis, said first tubular section having a first configuration and a second radially compressed configuration, said second tubular section forming a lumen;
a plurality of tubular branching members branching from said second tubular section, each of said tubular branching members having a first configuration and a second radially compressed configuration, each branching member forming a lumen that is in fluid communication with said lumen formed by said second tubular section; and
a plurality of sleeves, each one of said sleeves surrounding one of said first tubular section and plurality of tubular branching members and restraining said first tubular section and said plurality of tubular branching members in said first and second radially compressed configurations.

11. The prosthesis of claim 10 wherein each branching member forms a self-expanding stent graft.

12. The prosthesis of claim 11 wherein said first tubular section forms a self-expanding stent-graft.

13. The prosthesis of claim 10 wherein said first tubular section forms a self-expanding stent-graft.

14. The prosthesis of claim 10 wherein said first tubular section has length measured along said longitudinal axis of at least 50 mm.

15. A method of treating an aneurysm comprising:
advancing a tubular prosthesis having a restrained first self-expanding tubular section with a plurality of stents and a second tubular stent-less section with the first self-expanding tubular section in a radially compressed state through a vessel to an unexposed vessel location where the first self-expanding tubular section spans a target site;
unrestraining the first self-expanding tubular section to allow the first self-expanding tubular section to radially expand; and
securing the second tubular stent-less section of the tubular prosthesis to the vessel at a location where the vessel has been exposed.

16. The method of claim 15 wherein the first self-expanding tubular section is advanced through the aortic arch and into the descending aorta of a patient.

17. The method of claim 15 wherein the tubular prosthesis includes a plurality of tubes branching therefrom and each tube is advanced into one of the left subclavian, left common carotid, and brachiocephalic arteries.

18. The method of claim 17 wherein the stent-less section is secured to the ascending aorta.

19. The method of claim 16 wherein the second tubular stent-less section is sutured to the ascending aorta.

20. The method of claim 15 wherein the second tubular stent-less section is sutured to the vessel.
